(19) United States
(12) Patent Application Publication
(10) Pub. No.: US 2022/0187227 A1
(43) Pub. Date: Jun. 16, 2022

(54) METHOD OF ESTIMATING A MINERAL CONTENT OF A GEOLOGICAL STRUCTURE

(71) Applicant: EQUINOR ENERGY AS, Stavanger (NO)

(72) Inventor: Ketil HOKSTAD, Trondheim (NO)

(21) Appl. No.: 17/440,054

(22) PCT Filed: Apr. 21, 2020
(86) PCT No.: PCT/NO2020/050101

§ 371 Date: Sep. 16, 2021

(30) Foreign Application Priority Data
Apr. 29, 2019 (GB) 1905939.3

(51) Int. Cl. G01N 23/2251; G01V 1/38

(52) U.S. Cl. CPC G01N 23/2251; G01V 1/3852; G01V 2210/6161; G01N 2223/418; G01N 2223/616

(57) ABSTRACT

A method of estimating a mineral content of a seabed geological structure is provided wherein there is provided at least one geophysical parameter of the geological structure. The method includes inverting the at least one geophysical parameter to estimate the mineral content of the geological structure.

The present invention relates to the field of seabed mineral exploration. In particular, in relates to a method of estimating a mineral content of a geological structure, for example for seabed mineral exploration.

Seabed minerals such as metal sulphides can be valuable. However, mining for them, particularly in subsea locations, is difficult and expensive.

Valuable minerals, such as metal sulphides, are often found around so-called “black smokers”. If a black smoker is found, for example by chance, a rock sample may be taken from around the black smoker for analysis in a laboratory, to see whether sought-after minerals, or a sufficient (e.g. economically viable) level of such minerals, are present at that location. Valuable minerals, particularly around black smokers, are often present in much higher concentrations in sea-bed locations than in onshore locations. However, due to the cost and difficultly of finding seabed minerals, currently there is not known to be any offshore mining of seabed minerals.

There is therefore a need for an improved method of determining (or estimating) mineral content of a geological structure, particularly for use in offshore or seabed locations.

A first aspect of the present invention provides a method of estimating a mineral content of a seabed geological structure, wherein there is provided at least one geophysical parameter of the geological structure, the method comprising, inverting the at least one geophysical parameter to estimate the mineral content of the geological structure.

The inventors have discovered that a mineral content (e.g. a mineral concentration, for example by mass) may be estimated by inverting at least one geophysical parameter. Thus, a mineral content may be estimated based on a measurement of a geophysical parameter, or a geophysical parameter estimated from (e.g. by inversion of) measured geophysical data. At least some geophysical parameters may be estimated or determined without, for example, needing to perform a mining operation or take a sample for analysis at a laboratory. As such, this method can provide an easier, quicker and cheaper method of determining a mineral content of a geological structure.

The mineral content of the geological structure may be a concentration (or other measure of amount or quantity), for example, of a particular mineral or type (or group) of mineral(s) which may be present in the geological structure. Determining the mineral content of the geological structure does not necessarily mean determining the concentration of all minerals (or other elements or compounds) present in the geological structure. For example, determining a mineral content of the geological structure may mean determining the content (e.g. concentration) of a particular mineral or type (or group) of minerals(s) which may be present in the geological structure. For example, the mineral content may be a precious metal concentration of the geological structure. In a preferred embodiment, the mineral content of the geological structure is the metal sulphide or sulphate content (e.g. concentration) in the geological structure.

The method may comprise making a decision to explore (e.g. with exploration drilling), drill or mine the geological structure if the mineral content is estimated to be above a particular threshold. The value of the threshold above which a decision to explore, drill or mine the geological structure would be made may depend on factors such as the location and environment of the geological structure. For example, a decision to explore, drill or mine the geological structure may be made if the mineral content (e.g. metal sulphide content) is estimated to be above 2.5%, 3.0%, 3.5%, 4% or 5%. A lower threshold may be applied if the minerals to be mined (or explored) are more readily available, for example close to existing infrastructure and/or close to shipping lanes. On the other hand, if the minerals are located in a more distant or remote place, e.g. in which exploration, drilling or mining would be more difficult and/or expensive, then a higher threshold may be applied.

The method may then further comprise, once a decision has been made to explore, drill or mine the geological structure, actually exploring (e.g. by drilling), drilling or mining the geological structure. The geological structure may first be explored, e.g. by drilling, for example to check whether the mineral content determined by the above method is accurate, and/or what the actual mineral content is (for example by taking a sample of the geological structure for analysis in a laboratory). Then, if the exploration step determines (or results in the determination) that the mineral content is sufficiently high (e.g. to warrant mining, or to make mining economically attractive and/or viable), the method may further comprise actually mining the geological structure, e.g. for the mineral such as a metal sulphide.

As described in more detail below, the mineral content of the geological structure may be determined as a function of (preferably) horizontal and/or vertical position. For example, the mineral content may be determined at a series or (two dimensional or three dimensional) array of points over a particular area or region of a geological structure. The mineral content of the geological structure may be determined as an average over a particular horizontal area and/or vertical range (depth).

The term geological structure simply means a (e.g. particular) region of the subsurface, which may, for example, be of interest (e.g. have a high mineral content). A seabed geological structure is a geological structure beneath the sea (e.g. in the seabed).

Inverting or inversion is a well-known term in the art. It describes the process of calculating (or estimating), from at least one observed/measured parameter, the cause of the parameter (or at least one of the causes of the parameter). Thus, in the present case, physically speaking, the mineral content affects the geophysical parameter(s) of the geological structure. However, it is geophysical data that is(are) measured and not the mineral content. Calculating the mineral content from the geophysical parameter(s) may therefore be described as inverting.

The inversion may be considered to be a calculation that uses a model (such as phenomenological or rock physics model), such as discussed below. The model may relate the geophysical parameter(s) to the mineral content to calculate the mineral content value from the geophysical parameter(s).

The at least one geophysical parameter may comprise one or more of: electrical resistivity or conductivity, the induced polarisation coefficient, a magnetic parameter such as magnetization (e.g. total magnetization including both induced and remnant magnetization), density, p-wave velocity, and s-wave velocity.

Preferably, two or at least two geophysical parameters are used.

Preferably, the at least one geophysical parameter comprises at least one of the induced polarisation coefficient, magnetization (e.g. total magnetization) and density, and more preferably all three of these parameters. These parameters may be particularly useful in providing an estimate of (or constraining an estimate of) the mineral content of the geological structure as the mineral content of the geological structure can have a strong effect on the value of these parameters.

The at least one geophysical parameter may be determined from measured geophysical data, such as controlled source electromagnetic (CSEM) data, transient electromagnetic (TEM) data, magnetic data, magnetotelluric data, gravity data, and/or seismic data. For example, electrical resistivity or conductivity may be determined from CSEM data, the induced polarisation coefficient may be determined from TEM data, magnetization may be determined from magnetic data and/or magnetotelluric data, density may be determined from gravity data, and/or p-wave and/or s-wave velocity may be determined from seismic data.

Preferably, the at least one geophysical parameter is determined from measured geophysical data by inverting the measured geophysical data to determine the at least one geophysical parameter.

Thus, the method preferably comprises obtaining (or measuring) geophysical data, such as the geophysical data described above. The geophysical data may be obtained using (e.g. from) a vessel, e.g. a survey vessel, and/or with an automated underwater vehicle (AUV), for example.

In the present method, the magnetic parameter (if used) may be a magnetization of the geological structure, for example. The magnetization could be, and preferably is, the total magnetization of the geological structure, e.g. including both the remnant and induced magnetizations. Alternatively, just one of these magnetizations, e.g. the induced magnetization, could be used as the magnetic parameter.

The method preferably also comprises inverting and/or modelling to convert the measured geophysical data into the at least one geophysical parameter.

As mentioned above, the at least one geophysical parameter preferably comprises at least one of the induced polarisation coefficient, magnetization and density. As such, the measured geophysical data preferably comprises at least one of CSEM data, TEM data, magnetic data, and gravity data.

Magnetic data may comprise magnetic anomaly data, such as magnetic potential field data.

The at least one geophysical parameter, e.g. determined in this way from measured geophysical data, may then be inverted in order to determine the (a) mineral content of the geological structure.

The inversion of measured geophysical data to determine the at least one geophysical parameter may be performed using any standard geophysical inversion method. For example, it may be performed using a map inversion method, e.g. a Marquardt-Levenberg type map inversion method, or any other (e.g. 3D) inversion method serving the same purpose.

The inversion of measured geophysical data may determine the at least one geophysical parameter as a function of horizontal position and/or vertical position. The inversion of measured geophysical data may determine the at least one geophysical parameter as a function of horizontal position averaged over a (relevant) depth interval, for example.

The inversion of the at least one geophysical parameter to determine the mineral content of the geological structure is preferably performed using a Bayesian inversion method and/or a phenomenological (e.g. rock physics) model.

Metal sulphides (or other metal compounds or minerals) found in geological structures, for example, tend to be demagnetised due to hydrothermal alteration. As such, the phenomenological model which is used preferably describes the degree of (de)magnetisation of the geological structure as a function of the mineral content (e.g. metal sulphide content).

The model may also or alternatively describe the conductivity and/or polarisation (e.g. induced polarisation coefficient) as a function of the mineral content (e.g. metal sulphide content).

The phenomenological model may comprise one or more parameters, such as the initial titanium fraction of the lavas at the time of deposition and/or the total percentage of magnetic material in the subsurface. The one or more parameters may be calibrated, e.g. by combining the one or more parameters to form empirical factors that may be calibrated for, e.g. for each geological structure.

The method may comprise selecting a phenomenological model(s) that defines the relationship between the geophysical parameter(s) (e.g. conductivity, induced polarisation coefficient and/or magnetisation, and preferably all three of these) and the mineral content of the geological structure, e.g. for use in the inversion of the geophysical parameter to determine the mineral content of the geological structure.

The phenomenological model may be a relationship between the geophysical parameter(s) and the mineral content of the geological structure.

The phenomenological model may be selected based upon expected trends relating the geophysical parameter(s) to the mineral content of the geological structure. For instance, a geophysical parameter may generally increase or decrease (depending on the geophysical parameter) with increasing mineral content of the geological structure. For example, in the case of density being the geophysical parameter, density may increase with increasing mineral (e.g. metal sulphide) content; in the case of the induced polarisation coefficient being the geophysical parameter, the induced polarisation coefficient may increase with increasing mineral (e.g. metal sulphide) content; in the case of magnetization being the geophysical parameter, the magnetization may decrease with increasing mineral (e.g. metal sulphide) content; and in the case of conductivity being the geophysical parameter, the conductivity may increase with increasing mineral (e.g. metal sulphide) content.

When the geophysical parameter increases with increasing mineral content, the model may be a sigmoid function or exponential function or linear function.

When the geophysical parameter decreases with increasing mineral content, the model may be a decaying function, e.g. a decaying exponential function such as the Arrhenius equation (which may be used to describe the electric conductivity and/or resistivity of dry basalt, for example).

Thus, as can be understood from the above, the precise phenomenological model can be selected by the skilled person based upon knowledge of rock physics relations.

Preferably, the respective model relationships between mineral content and the at least one geophysical parameter are not dependent on any other variable, such as any other geophysical parameters. Of course, other constant factors may be present, but there is preferably only one variable. The constant factors may be found by calibration with data.

It should be understood that the phenomenological model may not show the full complexity of the system, i.e. the model may be intentionally simplified such that the geophysical parameter(s) (e.g. that/those selected for use in the method) is dependent only on the mineral content. In reality, geophysical parameters generally depend on many variables. However, in the model(s) used in the present method, the geophysical parameter(s) (e.g. that/those selected for use in the method) may only depend on the variable of interest, which in this case is mineral content. Thus, in the phenomenological model(s) used, preferably only the (geophysical) parameter(s) of interest for the statistical inversion are treated as stochastic parameters. Other (e.g. geophysical or other) parameters may be incorporated in the model as deterministic parameters with a fixed value.

There may be provided calibration data e.g. comprising at least one measurement of the geophysical parameter and the mineral content of the geological structure. This data may be taken, for example, from a (rock) sample of the geological structure. Thus, the method may further comprise obtaining calibration data. The calibration data may preferably contain a plurality of measurements of the geophysical parameter and the mineral content of the geological structure. In an example, well log data is used to calibrate and/or constrain the phenomenological model.

The method may comprise optimising the phenomenological model based on the calibration data. This optimisation may comprise using the calibration data to find the optimal values of the/any constant factors in the phenomenological model. Typically, the greater the amount of calibration data, the better the optimisation will be.

In order to optimise the phenomenological model, it may be assumed that the phenomenological model has a certain error distribution (i.e. the difference between the at least one geophysical parameter and the mineral content gives an error distribution). Preferably, the error distribution is assumed to be a Gaussian error distribution, preferably with zero mean. The phenomenological model may be optimised by reducing the error distribution so that it is as small as possible, such as by having a mean of the error distribution to be as close as possible to zero and by having a small a variance of the error distribution as possible. The optimisation may be achieved by finding the value(s) of the constant factor(s) in the phenomenological model that optimise(s) the phenomenological model.

The optimised phenomenological model may then be used in the inversion to produce a more accurate inversion.

The phenomenological model may be used in the inversion to calculate the probability distribution (and/or the mean and/or variance values (directly)) of the at least one geophysical parameter, given a particular value of the mineral content. This probability distribution function may be used to calculate the probability distribution of mineral content (and/or the mean and/or variance values (directly)), given particular values of the geophysical parameter.

By “phenomenological model” here, it may simply mean the mathematical relationships used in the inversion, such as the phenomenological model relating the geophysical parameter to the mineral content.

Performing the inversion in the Bayesian setting, as discussed above, can allow for an (accurate) estimate of the uncertainty in the calculated mineral content value to be found. Thus, the method may comprise finding the uncertainty in the calculated mineral content.

The geophysical data may be gathered using known techniques, such as with a magnetometer for magnetic data. A magnetometer may be carried by satellite, ship, or drone, for example. The method may comprise gathering/obtaining the geophysical data. For example, gravity geophysical data may be acquired from a ship, or an automated underwater vehicle (AUV); electromagnetic data may be acquired with receivers, e.g. measuring electric and magnetic fields, on the seabed, and an electric source at or close to the seabed; and seismic data may be obtained using a site-survey boat, a seismic 2D or 3D vessel, or using ocean bottom seismic receivers on the seabed, for example.

The geophysical data may have been (and preferably are) acquired prior to any mining operation, in which case the geophysical data may be considered to be pre-mining geophysical data. This data may be used to provide a pre-mining estimate of the mineral content (e.g. by performing the method of the present invention). Additionally or alternatively, the geophysical data may have been gathered during or after a mining operation. This may be used to provide a during-mining or post-mining estimate of the mineral content (e.g. by performing the method of the present invention), which may be considered to be an update to the existing estimates.

The method may comprise acquiring the geophysical data prior to mining. Additionally or alternatively, the method may comprise acquiring the geophysical data during mining. Additionally or alternatively, the method may comprise acquiring the geophysical data after a mining operation. The data acquired during or after mining may be or comprise mineral content data (i.e. a direct measurement of the mineral content), which could be used to update previous mineral content estimates (e.g. the pre-mining estimate, or a previous during-mining estimate) by offering a further constraint to the inversion problem.

The method may comprise calculating the mineral content prior to a mining operation of the geological structure.

Additionally or alternatively, the method may comprise calculating the mineral content during a mining operation. This may be considered to be an update to the pre-mining mineral content calculation.

Additionally or alternatively, the method may comprise calculating the mineral content after a mining operation. This may be considered to be an update to the pre-mining and/or during-mining mineral content calculation.

The above methods may calculate the mineral content for a specific point/location/volume/space of the geological structure, said point/location/volume/space corresponding to the point/location/volume/space of the (at least one) geophysical parameter used in the inversion step (the geophysical parameter(s) used in these methods may be the value of that parameter at a given point/location/volume/space in the geological structure). Therefore, in order to obtain a spatially dependent mineral content function, the above inversion method may be performed point-wise for multiple different points/locations/volumes/spaces in the geological structure. As can be appreciated, the geophysical parameter(s) may vary over the space of the geological structure, and this may correspond to a spatially varying mineral content.

Thus, the method may comprise constructing a spatially dependent mineral content function. This function may be constructed by calculating the mineral content for different points/locations/volumes/spaces in the geological structure (preferably all points/locations/volumes/spaces in the geological structure). The mineral content may be calculated over substantially the entirety of the geological structure, or over a particular area and/or depth.

The mineral content may be found in one, two or three dimensions.

The mineral content may be found as function of depth, and possibly also horizontal location. This may provide the user with an estimate of the depth, and possibly also the horizontal location, of possible targets for mining.

In some embodiments, the method comprises obtaining (e.g. measuring) first geophysical data of a first area of the geological structure and processing the first geophysical data to estimate the mineral content of the first area of the geological structure, preferably as a function of position (e.g. horizontal and/or vertical position). The method may then further comprise, e.g. if the mineral content of the first area of the geological structure is found to be greater than a particular value at any points or locations within the first area (i.e. in a second area), obtaining (e.g. measuring) second geophysical data of a second area of the geological structure and processing the second geophysical data to estimate the mineral content of the second area of the geological structure.

The first geophysical data are preferably obtained (measured) from a vessel such as a survey vessel. For example, the first geophysical data may comprise gravity and/or seismic data.

The second geophysical data may be obtained (measured) using an automated underwater vehicle. For example, the second geophysical data may comprise CSEM, TEM, magnetotelluric and/or magnetic data.

The second area is preferably an (smaller) area of the first area, e.g. in which the mineral content has been estimated as being above a particular level.

Processing the first and/or second geophysical data to estimate the mineral content of the first and/or second area of the geological structure preferably comprises determining one or more geophysical parameters from the first and/or second geophysical data, e.g. by inverting the first and/or second geophysical data, and then preferably inverting the one or more geophysical parameters to estimate the mineral content of the first and/or second area of the geological structure.

By obtaining and processing geophysical data in two (or more) stages in this way, this allows for first geophysical data to be obtained over a relatively large area, for example by using cheaper, quicker and/or easier techniques, e.g. from a survey vessel and then, only if an area of interest is discovered within the first relatively large area (i.e. with an estimated mineral content above a particular level and therefore warranting further investigation), obtaining and processing further (second) geophysical data over a smaller area of the first relatively large area, for example using more expensive and/or time-consuming techniques (e.g. with one or more AUVs), to improve the estimate of the mineral content for that second area.

In some embodiments, the method may comprise obtaining one or more geochemical parameters related to the geological structure and/or processing the one or more geochemical parameters to estimate the mineral content of the geological structure. Processing the one or more geochemical parameters preferably comprises inverting the one or more geochemical parameters to estimate the mineral content of the geological structure.

The geochemical parameters may be obtained from geochemical data, which may be obtained, for example, from rock samples (e.g. by analysing rock samples for example in a laboratory). The rock samples could be obtained by dredging the seabed/geological structure, and/or from water samples taken near the seabed/geological structure. The presence or concentration of certain signature minerals from hydrothermal alteration or gases (e.g. Helium 3He) in the sea water may be measured from one or more samples to provide one or more geochemical parameters.

As described above, the invention provides a method of estimating a mineral content of a geological structure. In a preferred embodiment, the metal sulphide content (e.g. concentration or fraction) is estimated as the mineral content. This would provide an estimate of the total metal sulphide concentration for the geological structure (or a point, area or region therein). The method may then further comprise obtaining a sample of geological structure and/or, preferably, determining (e.g. from that sample) which metal sulphide(s) is(are) present in the geological structure. For example, the method may comprise determining whether any or all of copper, zinc, silver or gold metal sulphide(s) is (are) present in the geological structure (e.g. from the sample) and, preferably, at what concentration.

In a preferred embodiment, the method may comprise checking or determining whether the metal sulphide concentration is above a particular threshold, such as described above.

The method could then comprise performing the steps of obtaining the sample (e.g. by drilling) and determining which metal sulphide(s) is(are) present in the geological structure only if the metal sulphide concentration is above the particular threshold. In this way, the (more expensive and difficult) step of obtaining a sample may only be performed if there is an indication that the metal sulphide concentration is sufficiently high to warrant further investigation.

If it is determined that one or more metal sulphides (e.g. copper, zinc, silver or gold metal sulphide(s)) is present in the geological structure at a significant concentration (e.g. above a particular threshold, such as 2.5%, 3.0%, 3.5%, 4.0% or 4.5%, and/or which may depend on other factors such as mentioned above and/or on the actual metal sulphide(s) which is/are determined to be present in the geological structure), the method may then further comprise mining the geological structure, e.g. for the one or more metal sulphides. As discussed, the threshold applied may (at least in part) depend on the actual metal sulphide(s) which is/are determined to be present in the geological structure. For example, more valuable metals such as silver or gold or rare earth elements may have a lower threshold applied than less valuable metals such as copper and zinc.

In a second aspect, the invention provides a method of producing a mineral content model of a geological structure comprising performing any of the methods of the first aspect.

As can be appreciated, the above methods may be used when prospecting for minerals (e.g. metal sulphides), e.g. when planning and performing mineral mining operations.

In a third aspect, the invention provides a method of prospecting for minerals comprising performing any of the methods of any of the first or second aspects and using the calculated mineral content in the decision-making process for the mining of a mine.

The calculated mineral content may be used prior to mining, e.g. when deciding where and/or how deep to mine the mine. The mineral content calculation may provide the user of the method with a mineral content (e.g. mineral content vs. depth) estimate, which can be used to decide where, how deep and/or in which direction to mine.

Additionally or alternatively, the calculated mineral content may be used during or after the mining of the mine, e.g. when deciding in which direction or to which depth to continue mining. This may particularly be the case where geophysical data is gathered during or after the mining of a mine, as discussed below.

The method may comprise performing a calculation of the mineral content prior to mining. This may be referred to as a pre-mining calculation. The pre-mining calculation may be used to decide where (and/or whether) to begin mining.

The method may comprise mining the mine.

The method may comprise acquiring new geophysical data during mining.

This new data may be for the same geophysical parameter(s) as were used in a pre-mining calculation. However, additionally or alternatively, this new data may be for different geophysical parameters to those which were used in the pre-mining calculation. For instance, the new data may additionally or alternatively comprise direct measurement of mineral content from within the mine. There may be new data for at least one, or preferably at least two, geophysical parameters. The new geophysical data may be gathered from the partly-mined mine, such as by taking one or more mine logs during mining.

The method may comprise inverting said new geophysical data to find corresponding new geophysical parameter(s). This may comprise performing the inversion from geophysical data to geophysical parameter as discussed above in relation to the first aspect. Where direct mineral content data has been acquired from the mine, this data may be used to constrain the inversion calculation.

The method may comprise inverting said new geophysical parameter(s) to provide updated estimates of the mineral content. Where there are two or more new geophysical parameters found during mining, these may be the only geophysical parameters used in the inversion. However, the new geophysical parameter(s) can also be inverted with one or more of the geophysical parameters used in the pre-mining calculation. This inversion step may comprise any of the features discussed in relation to the inversion of the geophysical parameters to estimate mineral content discussed in relation to the first or second aspects.

The steps of acquiring new geophysical data and inverting it to find updated estimates of the mineral content may be repeated throughout the mining process.

The updated estimate of the mineral content may be used during the mining process in the mining decision making process, such as deciding what direction to mine in and how deep to mine and whether to stop mining. Thus, a more educated mining process can be carried out using the present method.

In a fourth aspect, the invention provides a method of mining for minerals (e.g. metal sulphides) from a geological structure. This method may comprise performing any of the methods of any of the first, second or third aspects. The mining for minerals from a geological structure can be performed using any known mining technique.

In a fifth aspect, the invention provides a computer program product comprising computer readable instructions that, when run on a computer, is configured to cause a processer to perform any of the above methods.

Throughout the specification, terms such as “calculating” and “estimating” may be used. These are not intended to be limiting; rather they are merely meant to mean determining or obtaining a value for an actual physical value (or at least a (close) approximation or estimate of the physical value), such as mineral content (e.g. concentration).

Preferred embodiments of the invention will now be discussed, by way of example only, with reference to the accompanying drawings, in which:

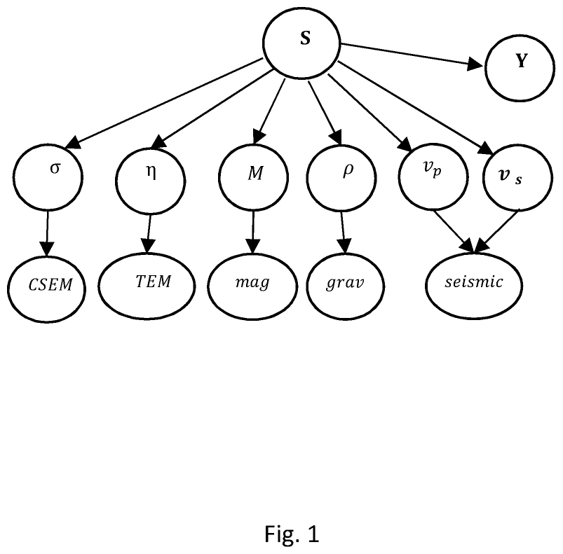

FIG. 1 is a general multi-geophysical Bayesian network for estimation of the mineral concentration S; and

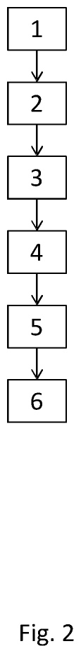

FIG. 2 is a flow chart illustrating a method of estimating a mineral content of a geological structure and mining for minerals.

As illustrated in FIG. 2, an embodiment of a method of estimating a mineral content of a geological structure and mining for minerals involves six main steps.

At step 1, geophysical data related to a geological structure is obtained over a relatively large subsea area, which has possibly been identified as being of potential interest, e.g. due to the presence of one or more black smokers.

As step 2, the geophysical data is processed to obtain an estimate of the mineral content, specifically the metal sulphide concentration, of the geological structure. The metal sulphide concentration is estimated as a function of horizontal position.

At step 3, if the metal sulphide concentration estimate is above a certain threshold (e.g. as 2.5%, 3.0%, 3.5%, 4.0% or 4.5%) at any locations in the geological structure, then more geophysical data is obtained for those locations. The threshold which is used is determined based on various factors including the economic viability of exploring and/or mining in that location, e.g. as discussed above.

At step 4, the new geophysical data obtained at step 3, possibly in combination with the geophysical data obtained at step 1, is processed to obtain a further (improved) estimate of the metal sulphide concentration of the geological structure as a function of horizontal position.

At step 5, if the further estimate of the metal sulphide concentration (determined at step 4) is above a certain threshold (e.g. as 2.5%, 3.0%, 3.5%, 4.0% or 4.5%) at any locations in the geological structure, then a sample of the geological structure at that/those locations is taken, by drilling, and analysed (e.g. in a laboratory) to determine which metal sulphides are present and at what concentration(s). Again, the threshold which is used is determined based on various factors including the economic viability of exploring and/or mining in that location, e.g. as discussed above.

At step 6, if a/any metal sulphide(s) of interest, e.g. copper, zinc, silver or gold metal sulphide(s), is (are) found to be present at sufficiently high concentration(s), e.g. above 2.5%, 3.0%, 3.5%, 4.0% or 4.5%, then a decision is taken to perform a mining operation for that/those metal sulphide(s) and the mining operation is subsequently performed.

Each of the above steps 1-6 will now be described in more detail.

At step 1, geophysical data related to a geological structure is obtained over a relatively large subsea area, such as up to 10,000 km². In some embodiments, the geophysical data collected at this step consists of only gravity, (possibly) magnetic and seismic data. This data can be collected using apparatus on board a survey vessel and there is no need, for example, to send an autonomous underwater vehicle (AUV) down to the seabed to collect other kinds of geophysical data.

However, in other embodiments, one or more of TEM data, magnetic data, CSEM data and magnetotelluric data are also or alternatively collected at this step, for example with EM receivers dropped from a vessel.

As step 2, the geophysical data is processed to obtain an estimate of the mineral content, specifically the metal sulphide concentration, of the geological structure. Step 2 actually contains two stages: at stage (i), the geophysical data collected at step 1 is inverted to determine geophysical parameters; and at stage (ii), the determined geophysical parameters are inverted to estimate the metal sulphide concentration of the geological structure.

This processing step is now explained in more detail with reference to FIG. 1.

Dependencies between physical quantities can conveniently be represented by Bayesian networks. FIG. 1 shows a general multi-geophysical Bayesian network for estimation of a mineral (e.g. metal sulphide) concentration S from geophysical parameters {σ, η, M, ρ, vₚ, vₛ}. As shown in FIG. 1, the geophysical parameters {σ, η, M, ρ, vₚ, vₛ} in turn depend on geophysical data, such as controlled source electromagnetic data (CSEM), transient electromagnetic data (TEM), magnetic data (mag), gravity data (gray), and seismic data (seismic), which can be included in an extended Bayesian network. In this figure, σ is resistivity (or conductivity), η is the induced polarisation coefficient, M is total magnetization (including both induced and remnant magnetization), ρ is density, vₚ is p-wave velocity, and vₛ is s-wave velocity.

Geochemistry parameter(s) Y may also be used to estimate the mineral concentration S. The geochemical parameters can be obtained by making laboratory measurements of rock samples and/or water samples. For example, the presence or concentration of certain signature minerals from hydrothermal alteration or gases (e.g. Helium 3He) in the sea water may be measured.

In principle, all of the variables in the Bayesian network of FIG. 1 can be regarded as stochastic. However, here, a simplified approach is used, taking only S and the actual measured geophysical data and the geophysical parameters on which they depend, as stochastic variables. The other variables are treated as deterministic hyperparameters or as having delta-function distributions.

Thus, in the case where just gravity and seismic data are collected, the main parameters of interest here are gravity data (gray), density (ρ), seismic data (seismic), p-wave velocity (vₚ), s-wave velocity (vₛ), and metal sulphide concentration (S).

The Bayesian network can be applied to obtain the joint distribution for a set of parameters, incorporating the principle of conditional independence. The joint probability of a set of stochastic nodes {x₁, . . . , xₙ} can be written as

$$p ( {x}_{1} , … , {x}_{n} ) = {Π}_{i} p ( {x}_{i} \vert {x}_{i}^{pa} ) , \qquad ( 1 )$$

where x^{pa}ᵢ denotes the parents of xᵢ, i.e. nodes on the level above in the network.

Using equation (1), and marginalizing hidden variables, the posterior distribution for metal sulphide concentration S given gravity and seismic data can be written as

p(S|d)=C∫p(S|m)p(m|d)dm (2)

where C is the normalization factor, m=(m₁, m₂, . . . , mₙ) is a vector of geophysical model parameters and d=(d₁, d₂, . . . , dₖ) is a vector of different geophysical data types, as discussed above.

The integral marginalizes the model parameters.

The geophysical model parameters mᵢ may be density, magnetization (induced and remnant), electric resistivity or conductivity, polarization coefficient, seismic P- and S-wave velocity.

The data dᵢ may be gravity data, magnetic data, electromagnetic data and seismic data.

As explained above, in practice, the inversion is performed in two separate steps:
(i) the (e.g. gravity and seismic) geophysical data are inverted to calculate the geophysical parameter(s) on which they depend (e.g. density ρ, p-wave velocity vₚ, and s-wave velocity vₛ); then
(ii) the geophysical parameters are inverted to determine the metal sulphide concentration S.

At step (i), the geophysical parameters density ρ, p-wave velocity vₚ, and s-wave velocity vₛ are computed by inversion of gravity and seismic data. Using Bayes rule, the following is obtained

p(m|d)=p(d|m)p(m) (3)

At step (ii), metal sulphide concentration S is computed by inversion of the geophysical parameters, e.g. density ρ, p-wave velocity vₚ, and s-wave velocity vₛ. Again using Bayes rule, the following is obtained

p(S|m)=p(m|S)p(S) (4).

This involves a non-linear phenomenological relationship between the geophysical parameters, e.g. density ρ, p-wave velocity vₚ, and s-wave velocity vₛ, and metal sulphide concentration S, which is discussed below.

Finally, the posterior distribution p(S|d) is obtained by means of equation (2). The marginalization of S can be written (in some cases) on a convolution form, which allows for fast and efficient computation using the fast Fourier transform (FFT).

Step (i) can be performed using standard, well-known geophysical inversion methods.

In some embodiments, step (i) is performed using a map inversion method, e.g. a Marquardt-Levenberg type map inversion method, to determine laterally varying geophysical parameters (density ρ, p-wave velocity vₚ, and s-wave velocity vₛ), each averaged over a relevant depth interval.

Alternatively, step (i) can be performed using any other (e.g. 3D) inversion method serving the same purpose.

Step (ii) uses a Bayesian inversion method involving a phenomenological model relating the geophysical parameter(s) (e.g. density ρ, p-wave velocity vₚ, and s-wave velocity vₛ) to the metal sulphide concentration, to calculate the metal sulphide concentration value from the geophysical parameter(s).

In order to obtain a spatially dependent 3D metal sulphide concentration function, the metal sulphide concentration of the geological structure is calculated point-wise for multiple different points/locations/volumes/spaces in the geological structure. As can be appreciated, the geophysical parameter(s) may vary over the space of the geological structure, and this may correspond to a spatially varying metal sulphide concentration.

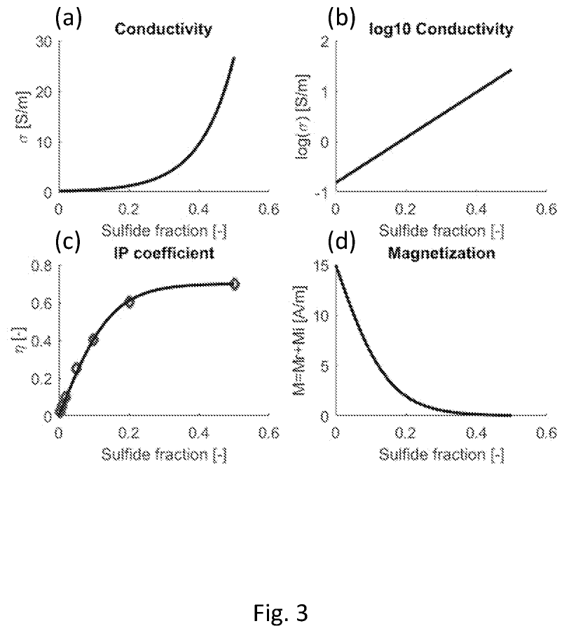

Phenomenological models which can be used to relate geophysical properties to underlying rock properties are illustrated in the charts in FIG. 3.

Charts (a) and (b) illustrates the relationship between conductivity σ and metal sulphide fraction. The logarithm of conductivity increases linearly with conductivity.

Chart (c) illustrates the relationship between the induced polarisation (IP) coefficient and the metal sulphide fraction. The induced polarisation (IP) coefficient increases with the metal sulphide fraction.

Chart (d) illustrates the relationship between the total magnetisation M (remnant and induced magnetisation) and the metal sulphide fraction. The total magnetisation decreases with the metal sulphide fraction.

Thus, following the above method, the metal sulphide concentration is estimated as a function of horizontal position.

Next, at step 3, if the metal sulphide concentration estimate is above a certain threshold such as described above at any locations in the geological structure (e.g. forming an area or region of interest), then more geophysical data is obtained for those locations (e.g. at the area or region of interest).

As discussed above, in some embodiments, the geophysical data collected at step 1 consists of only gravity and seismic data, which is collected from a survey vessel over an area of up to or around 10,000 km².

If step 2 indicates that there may be areas within that area which have sufficiently high metal sulphide concentrations to warrant further investigation, at step 3, more geophysical data is obtained for that (those) locations, e.g. within the area over which geophysical data was obtained at step 1.

In some embodiments, the geophysical data obtained at step 3 includes one or more of TEM data, magnetic data, CSEM data and magnetotelluric data. These kinds of data can be collected by sending an AUV down to the seabed at the area of interest.

The area or region of interest over which geophysical data is collected at step 3 is smaller than the area or region over which geophysical data is collected at step 1. For example, the area or region of interest over which geophysical data is collected at step 3 could be around 50 km².

At step 4, the new geophysical data obtained at step 3, possibly in combination with the geophysical data obtained at step 1 (e.g. for that area or region of interest), is processed to obtain a further (improved) estimate of the metal sulphide concentration of the geological structure as a function of horizontal position at the smaller area of interest.

The processing performed at step 4 follows the same stages (i) and (ii) as set out above in relation to step 2, the only difference being that more geophysical data and parameters are included in the calculations. Thus, the equations given above in relation to step 2 can be suitably modified to account for the geophysical data and parameters which are included at step 4.

As more geophysical data and parameters are included in the processing step to estimate the metal sulphide concentration, the better (more accurate) the estimate of the metal sulphide concentration becomes.

In some embodiments, steps 3 and 4 are omitted and all of the geophysical data that is used is obtained and then processed together in steps 1 and 2.

Next, at step 5, if the further estimate of the metal sulphide concentration (determined at step 4, or step 2 in some embodiments where steps 3 and 4 are not performed) is above a certain threshold such as described above at any locations in the geological structure, then a sample of the geological structure at that/those locations is taken, by drilling, and analysed (e.g. in a laboratory) to determine which metal sulphides are present and at what concentration(s).

At steps 2 and 4, only the total metal sulphide concentration is determined but not the concentration of (a) particular metal sulphide(s). Some metal sulphides are more valuable than others so it is important to check which metal sulphide(s) is(are) present in the geological structure, and at what concentration(s), before deciding whether or not to mine for it (them).

Thus, at step 5, a sample is taken from the geological structure from an area which has been determined to have a sufficiently high metal sulphide concentration to warrant further investigation. This sample is then tested in a laboratory to determine exactly which metal sulphides are present and at what concentration.

In some embodiments, step 5 involves determining whether any of all of copper, zinc, silver and/or gold metal sulphide(s) are present in the geological structure and at what concentration.

Finally, at step 6, if a/any metal sulphide(s) of interest, e.g. copper, zinc, silver or gold metal sulphide(s), is (are) found to be present at sufficiently high concentration(s), e.g. above 2.5-4%, then a decision is taken to perform a mining operation for that/those metal sulphide(s) and the mining operation is subsequently performed.

The above method can be used when prospecting for minerals (e.g. metal sulphides), e.g. when planning and performing mineral mining operations.

In one embodiment, the calculated mineral content (e.g. metal sulphide concentration) is used prior to mining, when deciding where to mine the mine and/or how deep to mine the mine.

In the same or other embodiments, the calculated mineral content is used during or after the mining of the mine, e.g. when deciding in which direction or to which depth to continue mining.

The mineral content estimate can be updated during mining based on new measured geophysical data.

I claim:

1. A method of estimating a mineral content of a seabed geological structure, wherein there is provided at least one geophysical parameter of the geological structure, the method comprising, inverting the at least one geophysical parameter to estimate the mineral content of the geological structure; and wherein the at least one geophysical parameter is determined from measured geophysical data by inverting the measured geophysical data to determine the at least one geophysical parameter.

2. As method as claimed in claim 1, wherein the mineral content of the geological structure is determined as a function of horizontal and/or vertical position.

3. A method as claimed in claim 1, wherein the at least one geophysical parameter comprises one or more of: electrical resistivity or conductivity, the induced polarisation coefficient, a magnetic parameter, density, p-wave velocity, and s-wave velocity.

4. A method as claimed in claim 1, wherein at least two geophysical parameters are used.

5. A method as claimed in claim 1, wherein the at least one geophysical parameter comprises at least one of the induced polarisation coefficient, magnetization and density.

6. A method as claimed in claim 1, wherein the geophysical data comprises CSEM data, TEM data, magnetic data, magnetotelluric data, gravity data, and/or seismic data.

7. A method as claimed in claim 1, further comprising obtaining the geophysical data.

8. A method as claimed in claim 1, wherein inverting the at least one geophysical parameter to estimate the mineral content of the geological structure comprising using a Bayesian inversion method and/or a phenomenological model.

9. A method as claimed in claim 8, wherein the method comprises selecting one or more phenomenological models that define the relationship between the at least one geophysical parameter and the mineral content of the geological structure.

10. A method as claimed in claim 1, wherein the mineral content of the geological structure is estimated prior to performing a mining operation of the geological structure and/or during a mining operation of the geological structure and/or after a mining operation of the geological structure.

11. A method as claimed in claim 1, the method comprising inverting the at least one geophysical parameter point-wise to estimate the mineral content of the geological structure for multiple different points/locations/volumes/spaces in the geological structure.

12. A method as claimed in claim 1, the method comprising:
(a) obtaining first geophysical data of a first area of the geological structure and processing the first geophysical data to estimate the mineral content of the first area of the geological structure; and then
(b) obtaining second geophysical data of a second area of the geological structure and processing the second geophysical data to estimate the mineral content of the second area of the geological structure.

13. A method as claimed in claim 12, wherein step (b) is only performed if the mineral content of the first area of the geological structure is found to be greater than a particular value at any point or location within the first area.

14. A method as claimed in claim 12, wherein the first geophysical data are obtained from a vessel and/or comprise gravity and/or seismic data.

15. A method as claimed in claim 12, wherein the second geophysical data are obtained using an automated underwater vehicle and/or comprise CSEM, TEM, magnetotelluric and/or magnetic data.

16. A method as claimed in claim 12, wherein the second area is a smaller area of the first area.

17. A method as claimed in claim 12, further comprising obtaining one or more geochemical parameters related to the geological structure and processing the one or more geochemical parameters to estimate the mineral content of the geological structure.

18. A method as claimed in claim 1, wherein the mineral content of the geological structure is the metal sulphide content of the geological structure

19. A method as claimed in claim 18, further comprising obtaining a sample of geological structure and/or determining which metal sulphide(s) is(are) present in the geological structure.

20. A method as claimed in claim 1, further comprising making a decision to mine the geological structure if the mineral content is estimated to be above a particular threshold.

21. A method of prospecting for minerals comprising performing the method of claim 1 and using the estimated mineral content in the decision-making process for the mining of a mine.

22. A method as claimed in claim 21, further comprising mining the geological structure.

23. A computer program product comprising computer readable instructions that, when run on a computer, is configured to cause a processer to perform the method of claim 1.
